(19) United States
(12) Patent Application Publication
(10) Pub. No.: US 2004/0209253 A1
(43) Pub. Date: Oct. 21, 2004

(54) SNP-based HLA-DP, DR and DQ genotyping analysis by reversed dot blot flow through hybridization

(76) Inventor: Joseph Wing On Tam, Daly City, CA (US)

Correspondence Address: Albert Wai-Kit Chan, Law Offices of Albert Wai-Kit Chan, LLC, World Plaza, Suite 604, 141-07 20th Avenue, Whitestone, NY, 11357, US

(21) Appl. No.: 10/291,168

(22) Filed: Nov. 7, 2002

Related U.S. Application Data
(60) Provisional application No. 60/345,948, filed on Nov. 7, 2001.

(51) Int. Cl. C12Q 1/68; C12P 19/34; C12M 1/34

(52) U.S. Cl. 435/6; 435/287.2; 435/91.2

(57) ABSTRACT

The present invention disclosed the use of Allele-Specific-Oligonucleotide (ASO) as the detection assay for human HLA classification. Using the Reversed-Dot-Blotting format and the Flow Through Hybridization process, more efficient, fast and less expensive HLA classification can be achieved. The simple procedures for the process were described. This invention can also provides the method for genotyping as well as DNA analyses in general for different genes and different organisms.

### CROSS REFERENCE

This is a regular application of a provisional application, application No. 60/345,948, filed on Nov. 7, 2001.

### BACKGROUND OF THE PRESENT INVENTION

1. Field of Invention

The present invention relates to method of making definitive identification of a human leukocytic antigens (HLA) by DNA analysis and the device thereof.

2. Description of Related Arts

Accurate HLA typing is essential for matching the donor and the recipient in organ or marrow transplantation (Thomas ED (1983) J. Clinical Oncology 1, 517-531) to prevent the development of acute graft-versus-host disease (GVHD) from unrelated donor. This is generally done by standard serological typing (Mach B et al. (1990) in Molecular Biology of HLA Class II Antigens, ed. Silver J (CRC, Boca Raton, Fla.), pp. 201-223). Recent studies have demonstrated that the use of DNA genotyping can provide more accurate and definitive result. The results of HLA-DQ, DR and DP). provide evidence for accurate matching necessary in selecting potential donors. In this case using sequence-specific primers polymrase chain reaction (SSP-PCR) amplifications have been reported for the purpose (Bunce M et al. (1995) Tissue Antigens, 46, 355-367). However, DNA sequencing is still considered the method of choice for accurate genotyping the HLA cluster. Unfortunately, because of the existence of highly homologous sequence of pseudgene(s) that may be co-amplified during the PCR amplification process, accurate genotyping by DNA sequencing alone may prove more difficult and costly. Our patented Direct Flow-through DNA Hybridization (Tam JWO, U.S. Pat. No. 5,471,547 (1998) & U.S. Pat. No. 6,020,187(2000)) is the fastest annealing process that uses a very inexpensive device for accurate mutation detection, genotyping and fingerprinting analysis. The present invention applied here presents the data of analyzing the HLA loci of DP, DR and DQ beta sequences by ASO oligo-probes using the Flow-through format.

### SUMMARY OF THE PRESENT INVENTION

### DETAILED DESCRIPTION OF THE PRESENT INVENTION

Our preliminary results suggested that the Allelic-Specific-Oligonucleotide Reversed-Dot-Blotting (ASO-RDB) direct Flow-through Hybridization is a good alternative for the detection of specific target HLA DNA sequences. Our data so far obtained refer to the specific segments of HLA loci of DP, DR and DQ beta that are able to provide accurate determination of the genotypes. Using one pair of PCR primer and 35 ASO oligo-probes, we can effectively classify the 83 DPB₁ alleles identified by the WHO four digit codes. Similarly, using one common PCR primer pair, and 18 ASO oligo-probes each, this simple hybridization protocol can identify the first 2 digit codes of the specific genotypes of the DR and DQ beta loci, enough to distinguish major classes of the HLA. However, when we use the same PCR primer pairs to perform the direct sequencing on the DR and DQ loci, un-interpretable sequencing data occurred frequently because of the co-amplification of pseudogene fragments. Hence, when we tried to confirm our ASO data (all samples were confirmed) we have to create many specific sets of PCR primers for which PCR amplification done in separate reactions. The positive amplicon(s) were then sequenced. This is why direct sequencing may prove to be costly. Although further detailed classification of the DR, DQ subtypes requires additional oligo-probes using the Direct Flow-through method, the number of such oligo-probes are well within the capability of the present format. This alternative method for HLA typing is faster, simpler, requires no expensive equipment and therefore much less costly compared to DNA sequencing.

The primers and oligo-probes shown in the sequence listing attached have been tested and confirmed to be useful for the number of loci reported above. Although these are not yet able to identify all the other HLA genotypes of all classes, using the scheme presented in FIG. 1, additional ones can be selected, tested and validated for full genotyping in the future. This is in the process of being tested. Although we use PCR as the amplification method but any other method that can produce specific target sequences in enough quantity and/or without amplification if naturally occur in high enough quantity for testing can also be used for the ASO-RDB flow through Hybridisation determination. The detection can be by labeling of the target DNA or conjugates through any known or those developed in the future appropriate for present method of detection.

Although HLA is used as the practical example here, this SNP-based analysis method can be applied the other gene and sequences of any organism follow the general procedures as shown in FIG. 1. The Flow-through Device can be those described in the U.S. Pat. Nos. 5,741,547 or 6,020,187 or any new embodiments capable of carrying out the said Flow-through Hybridization process.

Although more data may be needed for forensic validation, this SNP-based Flow Through format has proven to be a good and accurate alternative for human identification. In addition to data already accumulated and analyzed, expansion of the Data Base can easily be accumulated.

### BRIEF DESCRIPTION OF THE DRAWINGS

FIG. 1 is the diagram of the method for obtaining the ASO probes and PCR primers data base of the invention.

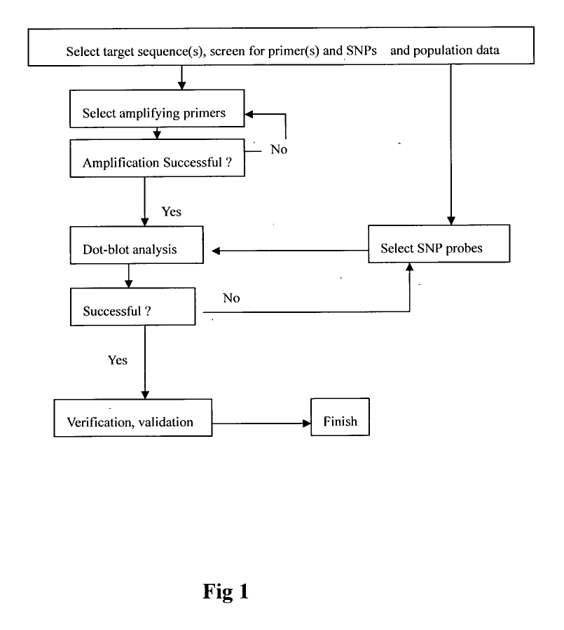

FIG. 2 is one of the example data for HLA-DRB and DQB genotyping identification.

FIG. 3 is one of the example data for HLA-DPB genotyping identification

FIG. 4 is the HLA-DQB genotype ASO detection profile

FIG. 5 is the HLA-DPB1 genotype ASO detection profile

Table 1 is the HLA-DPB1 gene typing results for Chinese

Table 2 is the HLA-DPB1 allele and genotype frequencies results for Chinese

### DETAILED DESCRIPTION OF THE METHOD OF THE INVENTION

The following is the general procedure for the present invention:

(a) Select gene segments and determine the PCR primers and the ASO sites:

1 Select the appropriate the specific target sequences to be analyzed by either screening data from the GenBank and/or perform population screening by sequencing the target genes or target DNA segments to get the SNP (or ASO) profile related to the HLA genotyping.

2 From these data determine the ASO sites to be used for genotyping based on the population data to evaluate if the sites are indeed unique for HLA typing within a population from Genbank or generated by sequencing the random sampling.

3 Then determine the number of ASO capture probes.

(b) Perform ASO-RDB detection for genotyping:

1 Immobilizing the ASO oligonucleotides onto the membrane or any matrix for capturing the target loci.

2 Then amplification the target segments using the appropriate primers by labelling the amplified target DNA molecules for the ASO-probes for hybridization detection.

3 Perform the ASO profile analyses by Flow-through Hybridization process using the device depicted in U.S. Pat. No. 6,020,187. The hybridisation is done in the hybridisation chamber with the ASO capture probes in the membrane:

a) Denature and drop the target DNA solution onto the membrane; wash and

b) Develop the color for visual inspection or spectrometric measurements (The target DNA can be labeled with flourencence dye in which case direct spectrometric determination other than color development by enzyme linked immuno-specific assay is needed).

4 Then the results are compared with known sequence data for accuracy evaluation.

5 Modify the probes and testing conditions for accuracy. The RDB-ASO data are verified by DNA sequencing.

(c) Validation

Proceed for validation with random samples.

### EXAMPLE

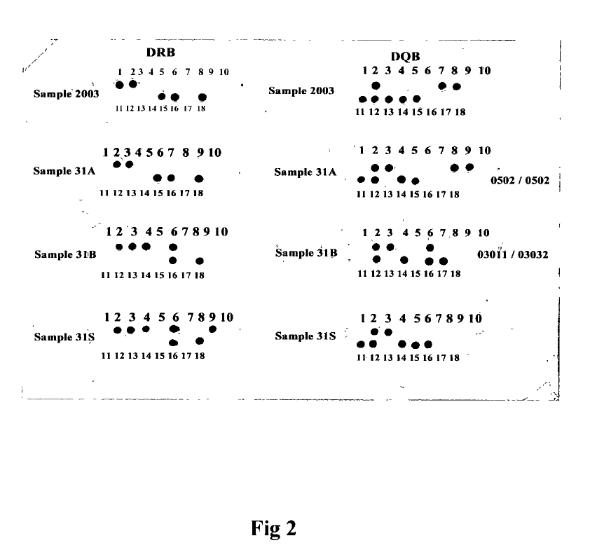

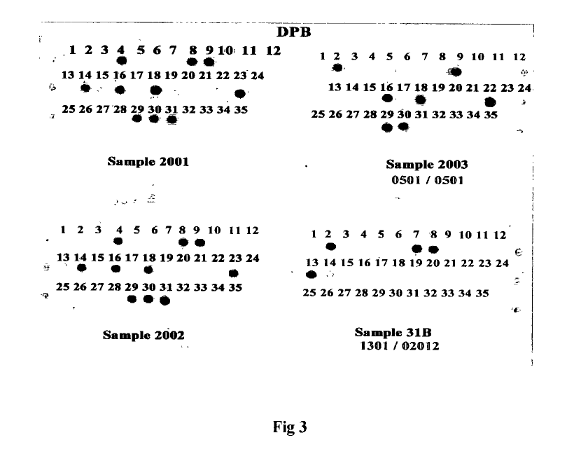

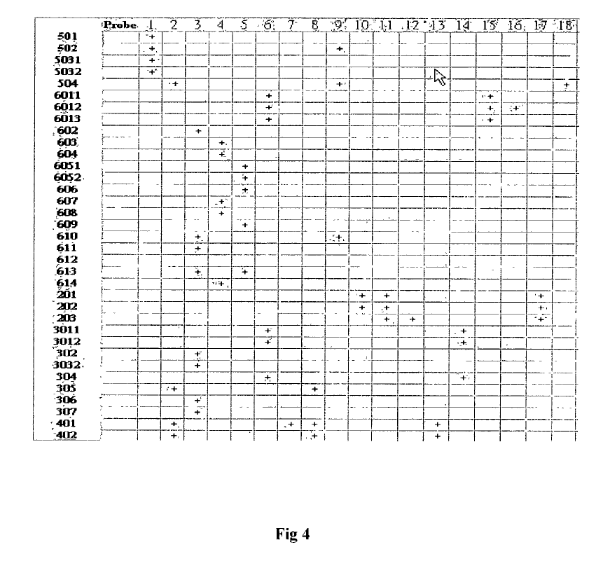

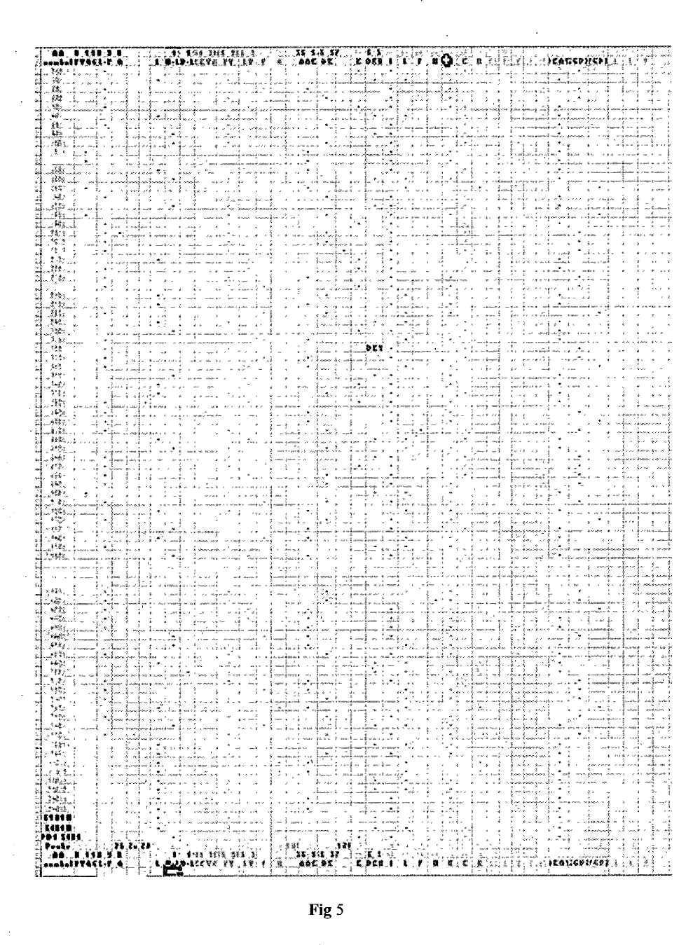

We have designed and evaluated 109 ASO probes for the HLA cluster and four PCR primers for amplification. FIG. 2 showed the typical results of the HLA-DRB and DQB loci and the classification into genotypes. FIG. 3 is the results for the HLΛ-DPB1. The ASO_RDB data summary are given in FIG. 4 and 5 and the genotypes and allele frequencies are given in Table 1 and 2. All data were confirm with DNA sequencing determination. In principle, any known ASO or SNPs of any organisms with adequate data to perform genetic analysis can be tested or detected by the Flow-through Hybridization Method. The results of hybridisation is done within minutes.

Reference

1 Bunce M et al. (1995) Tissue Antigens, 46, 355-367 Mach B et al. (1990) in Molecular Biology of HLA Class II Antigens, ed. Silver J (CRC, Boca Raton, Fla.), pp. 201-223

2 Mach B et al. (1990) in Molecular Biology of HLA Class II Antigens, ed. Silver J (CRC, Boca Raton, Fla.), pp. 201-223

3 Tam Joseph Wing On, Flow Through Nucleic Acid Hybridisation Device, U.S. Pat. No. 6,020,187.

4 Thomas ED (1983) J. Clinical Oncology 1, 517-531)

What is claimed is:

1. A flow-through hybridization method for nucleic acid assay for determination of the identification of an organism comprising:
a the flow-through hybridization device;
b. a matrix material capable of immobilizing the Allel-Specific-Oligonucleotide (ASO) probes for capturing the target DNA for analysis;
c. A set of ASO probes as given in the DNA sequence listing in this invention for capturing the target DNA molecules to be tested;
d. A set of PCR primers as given in the DNA sequence listing in this invention for amplifying the target DNA segments to be tested;
e. A set of amplified target DNA molecules are linked with labeled capable of generating signal for monitoring and detection.

2. The hybridization device according to claim 1, whereas the device can be any kind comprising a hybrydisation chamber, a matrix and the device for liquid flow through process directing through the matrix for capturing the target molecules;

3. The matrix according to claim 1, whereas the matrix can be any material capable of the immobilizing the ASO probes for capturing target DNA;

4. The ASO probes according to claim 1, are HLA specific as listed but can be any oligonuleotide sequence determined from the genome of the organism having specificity for said gene and the said population;

5. The ASO probes according the claim 1, whereas the number of ASO oligonucleotides can be any number in combination needed to achieve definitive detection for classification and identification;

6. The DNA molecules according to claim 1, whereas the DNA molecules is the target DNA segments;

7. The labeling molecules according to claim 1, whereas the label molecules are biotin or dioxigennin or radioactive isotopes.

8. The labeling molecules according to claim 1, whereas this label molecule can be any kind that is by itself or in combination with other material capable of generating signal for detection;

9. The signal generating molecules according to claim 8, whereas the molecules can enzyme linked specific antibodies or magnetic particles linked with antibodies or colloidal gold linked with specific antibodies or ligand receptors capable of generating signal;
